(19) United States
(12) Patent Application Publication
(10) Pub. No.: US 2013/0237958 A1
(43) Pub. Date: Sep. 12, 2013

(54) NEEDLE STYLET AND METHODS OF IMPLEMENTATION AND MANUFACTURE

(71) Applicant: Anthony C. ARRIGO, North Andover, MA (US)

(72) Inventor: Anthony C. ARRIGO, North Andover, MA (US)

(21) Appl. No.: 13/789,299

(22) Filed: Mar. 7, 2013

Related U.S. Application Data
(60) Provisional application No. 61/608,276, filed on Mar. 8, 2012.

(51) Int. Cl. A61M 25/01; B21G 1/00; A61B 17/34

(52) U.S. Cl. CPC A61M 25/0102; A61B 17/3401; B21G 1/00
USPC 604/506; 604/164.01; 163/5

(57) ABSTRACT

A needle apparatus and a method of implementing a needle apparatus are disclosed. The needle apparatus includes a hollow tube component including, at a first end of the hollow tube component, a pointed portion configured for piercing tissue of a patient and a needle hub disposed at a second end of the hollow tube component. The hollow tube component includes a bend disposed therein. The bend is positioned between the first and second ends of the hollow tube component. The needle apparatus also includes a stylet removably positioned in the hollow tube component. The stylet is characterized by a proximal end and a distal end. The stylet includes a stylet hub coupled to the distal end. At least a portion of the proximal end of the stylet includes a first diameter that is smaller than a second diameter of at least a portion of the distal end of the stylet.

### CROSS REFERENCE TO RELATED APPLICATIONS

The present application claims priority to U.S. Provisional Application No. 61/608,276, filed Mar. 8, 2012, entitled “REMOVABLE STYLET FOR BENT PAIN MANAGEMENT SPINAL NEEDLES,” which application is incorporated herein by reference in its entirety.

### TECHNICAL FIELD

The present embodiments relate generally to a needle apparatus and methods of implementing and manufacturing the same. Disclosed exemplary embodiments relate more specifically to a spinal needle for receiving a stylet and a method of making the stylet. The stylet may be introduced into the spinal needle when the spinal needle enters a patient's body.

### BACKGROUND

During medical procedures, such as surgical procedures to the vertebrae and other parts of the spine, a spinal needle may be used. The spinal needle may be used to collect cerebrospinal fluid for analysis or to relieve pressure. Alternatively, the spinal needle may be used to deliver medicine. A tip of a spinal needle that enters a patient's body may be disposed at an angle relative to the rest of the spinal needle. A stylet may be inserted into the spinal needle with the angled tip when the spinal needle enters the body to prevent undesired tissue from entering the spinal needle. Conventional stylets may be made of metal or plastic.

Disadvantages of conventional spinal needles may include the difficulty in removing a stylet after the spinal needle is positioned at a desired location within a patient's body. The difficulty increases when both the spinal needle and the stylet are made of metal because any clearance between the stylet and the spinal needle, at the angled or bent portion of the spinal needle, is small.

### SUMMARY

The inventor has appreciated that inventive embodiments disclosed herein provide a needle apparatus and methods of implementing the same that reduce difficulties associated with removing a stylet from a needle having a bent or angled portion.

Accordingly, various exemplary embodiments disclosed herein provide a needle apparatus. The needle apparatus includes a hollow tube component including, at a first end of the hollow tube component, a pointed portion configured for piercing tissue of a patient and a needle hub disposed at a second end of the hollow tube component. The hollow tube component includes a bend disposed therein. The bend is positioned between the first and second ends of the hollow tube component. The needle apparatus also includes a stylet removably positioned in the hollow tube component. The stylet is characterized by a proximal end and a distal end. The stylet includes a stylet hub coupled to the distal end. At least a portion of the proximal end of the stylet includes a first diameter that is smaller than a second diameter of at least a portion of the distal end of the stylet.

In various embodiments, the portion of the proximal end of the stylet that includes the first diameter has a length that is greater than a distance from the pointed portion of the hollow tube component to the bend. In various embodiments, the portion of the proximal end of the stylet including the first diameter has a length of at least a distance from the pointed portion of the hollow tube component to the bend.

The second diameter corresponds to an inner diameter of the hollow tube component, in accordance with some embodiments.

The stylet may include a pointed tip positioned at the proximal end.

The stylet may be composed of stainless steel.

In various embodiments, an inner diameter of the hollow tube component is substantially constant from the first end to the second end.

A diameter of the hollow tube component is smaller at the first end than at the second end, in various embodiments.

In various embodiments, the hollow tube component and the stylet are composed of the same material.

In various embodiments, the hollow tube component and the stylet are composed of distinct materials.

Various exemplary embodiments provide a method of implementing a needle apparatus. The method includes positioning a stylet in a tubular needle component. The stylet is characterized by a proximal end and a distal end. The stylet includes a stylet hub coupled to the distal end. At least a portion of the proximal end of the stylet includes a first diameter that is smaller than a second diameter of at least a portion of the distal end of the stylet. The needle apparatus includes a hollow tube component including, at a first end of the hollow tube component, a pointed portion configured for piercing tissue of a patient and a needle hub disposed at a second end of the hollow tube component. The hollow tube component includes a bend disposed therein. The bend is positioned between the first and second ends of the hollow tube component. The needle apparatus also includes a stylet removably positioned in the hollow tube component. The method further includes removing the stylet from the tubular needle component.

Various exemplary embodiments provide a method of manufacturing a stylet for a needle apparatus. The method includes removing material from the stylet such that at least a portion of a proximal end of the stylet includes a first diameter that is smaller than a second diameter of at least a portion of a distal end of the stylet. The method further includes coupling a stylet hub to the distal portion of the stylet.

In various embodiments, removing material includes removal by at least one of grinding, milling, and electrical discharge machining.

The method includes forming a pointed tip on the proximal end of the stylet, in accordance with various embodiments.

In various embodiments the method includes removing material from the stylet such that the portion of the proximal end of the stylet including the first diameter has a length greater than a distance from a pointed portion of a hollow tube of the needle apparatus to a bend in the hollow tube of the needle apparatus.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is a top view of spinal needle with a stylet disposed therein in accordance with an exemplary inventive embodiment.

FIG. 2 is a cross-sectional view of the spinal needle of FIG. 1 at section 60, with the stylet removed.

FIG. 3 is a cross-sectional view of a portion of the stylet of FIG. 1.

FIG. 4 is a cross-sectional view of the spinal needle of FIG. 1 at section 60, with the stylet in position.

The features and advantages of the inventive concepts disclosed herein will become more apparent from the detailed description set forth below when taken in conjunction with the drawings.

### DETAILED DESCRIPTION

Following below are more detailed descriptions of various concepts related to, and embodiments of, inventive apparatuses and methods for a needle apparatus. The disclosure relates to needle apparatuses configured to receive a stylet and methods of making and using the stylet with needles. The stylet may be introduced into a needle before or after the needle enters a patient's body. The stylet may be used in needles such as spinal needles or other needles. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

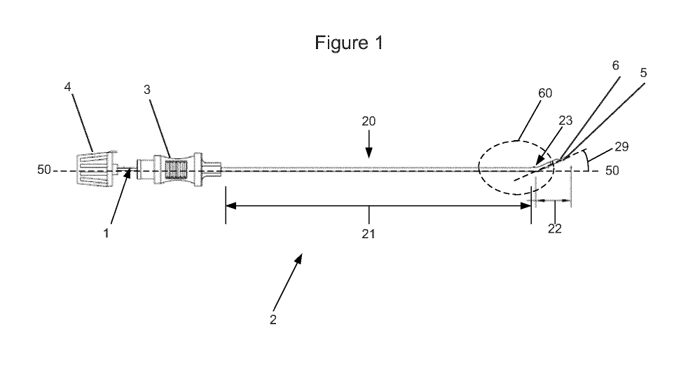

Referring generally to the Figures, various embodiments of a needle apparatus are shown. FIGS. 1-4 illustrate a spinal needle 2 with a stylet 1 therein.

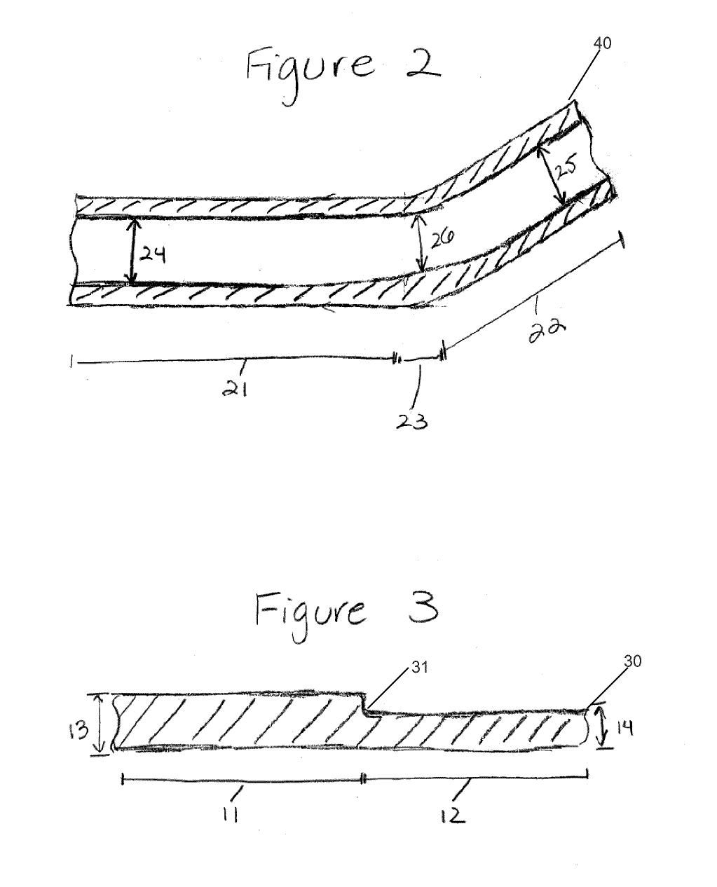

The spinal needle 2 (FIGS. 1-2) may include a hollow tube component 20. Hollow tube component 20 may include a tube first portion 21, a tube second portion 22 and a tube connecting portion 23 that connects the tube first portion 21 to the tube second portion 22. Preferably, the tube first portion 21, the tube second portion 22 and the tube connecting portion 23 are collectively formed from a single, integral, tube. The tube first portion 21 extends along a longitudinal axis 50-50. The tube second portion 22 is at an angle 29 relative to the longitudinal axis 50-50. The tube connecting portion 23 is located at the portion of the hollow tube 20 where the hollow tube bends at an angle 29. Needle 2 may also include a needle hub 3 configured for handling the needle and or introducing substances or components into the hollow tube 20. Needle 2 may also include a pointed portion or bevel 5 configured for piercing tissue of a patient upon insertion of needle 2 into the tissue of the patient.

In a manner similar to needle 2, stylet 2 may also include a hub, namely stylet hub 4 positioned at a distal end of stylet 2 and configured for handling the stylet, for example for positioning the stylet into tube 20 and removing stylet 2 from tube 20. Stylet 2 may also include a pointed tip portion or bevel 6 at a proximal end of the stylet, which pointed portion may be shaped to correspond to portion 5 of needle 2. Because hollow tube 20 is hollow, stylet 2 may be positioned in tube 20, to assist in the prevention of tissue, bodily fluid, or other substances into tube 20 upon insertion of tube 20 into the patient.

The inner portion of the tube first portion 21, the inner portion of tube second portion 22, and the inner portion of the tube connecting portion 23 each have inner diameters 24, 25, and 26 respectively (FIG. 2). In some embodiments, inner diameters 24, 25, and 26 are substantially the same. Diameter 26 may be slightly smaller in some embodiments because of the bend. In some embodiments, inner diameter 24 and inner diameter 25 may be distinct.

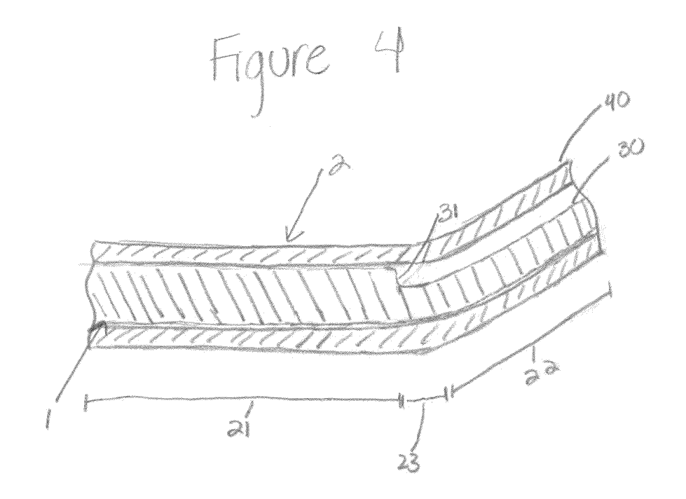

The stylet 1 may include a stylet first portion 11 and a stylet second portion 12 (FIG. 3). The stylet first portion 11 has a stylet first diameter 13 and the stylet second portion 12 has a stylet second diameter 14 that is smaller than the stylet first diameter 13. The stylet first diameter 13 and the stylet second diameter 14 are the outer diameters of the stylet first portion 11 and the stylet second portion 12, respectively. In some embodiments, the stylet second portion 12 may have a circular cross section. In some embodiments, the stylet second portion 12 may have a semi-circular cross section. In embodiments where hollow-tube 20 has a non-circular geometry and/or the inner hollow channel has a non-circular cross-section, stylet first portion 11 and stylet second portion 12 may have non-circular geometries, which correspond in shape to the shape of the inner hollow channel of hollow-tube 20. As used herein diameter, while generally describing the width of a circular cross-section, may encompass the width or height of a non-circular cross section of the inner diameter of a hollow needle or stylet first or second portion, exclusive of the beveled tip or pointed portion of the stylet.

The hollow tube 20 of the spinal needle 2 is configured to receive the stylet 1 (FIG. 4). For the stylet 1 to fit within the hollow tube 20, the first stylet diameter 13 and the second stylet diameter 14 must be smaller than the tube first inner diameter 24 and the tube second inner diameter 25. Additionally, the stylet second diameter 14 preferably is smaller than the tube connecting inner diameter 26. When the stylet 1 is aligned with the hollow tube 20, such that the end 30 (FIGS. 3-4) of the stylet second portion 12 is flush with the end 40 (FIGS. 2 and 4) of the tube second portion 22, the beginning 31 (FIGS. 3-4) of the stylet second portion 12 begins before the tube connecting portion 23 because the stylet second portion 12 is longer than the distance from end 40 to the bend of section 23. Consequently, the hollow tube 20 can easily receive the stylet 1, an operator can easily remove the stylet 1 from the hollow tube 20 and the stylet first diameter 13 can help prevent undesired tissue from entering the spinal needle 2 when the spinal needle 2 enters a patient's body.

The spinal needle 2 and the stylet 1 may be made of any suitable material. For example, both the spinal needle 2 and the stylet 1 may comprise the same material (e.g. metal including, but not limited to, steel, stainless steel, etc.). When the spinal needle 2 and the stylet 1 comprise the same material, it is easier to remove the stylet 1 from the spinal needle 2 and prevent unwanted tissue from entering the spinal needle 2 when the stylet second diameter 14 is smaller than the stylet first diameter 13 so that the stylet second portion 22 does not get caught at the tube connecting portion 23. In some embodiments, spinal needle 2 may comprise one type of material and the stylet 1 may comprise a different material.

A method of making the stylet 1 for the spinal needle 2 may include removing material from the stylet 1 at the stylet second portion 12 so that the stylet second diameter 14 is smaller than the stylet first diameter 13 (or such that width of the cross-section, in the case of a non-circular stylet, of the second portion is smaller than the width of the cross-section of the first portion 11). Before removing the material, the stylet first diameter 13 is equal to or substantially equal to the stylet second diameter 14. Any suitable mechanism may be implemented to remove the material from the stylet 1 at the stylet second portion 12, thereby providing the stylet with a relief. For example, the material may be removed by grinding, milling, or electrical discharge machining (EDM). The surface texture of the stylet second portion 12 after the material is removed may be smooth or rough, but the latter is not preferred. The transition from the stylet first portion 11 to the stylet second portion 12, after the material is removed, may be smooth (e.g. rounded) or rough, but the latter is not preferred. The stylet first portion 11 and the stylet second portion 12 could be integrally formed. Alternatively, the stylet first portion 11 and the stylet second portion 12 could be separate components joined or coupled together, but this is not preferred. The stylet hub 4 may be coupled to stylet first portion 11 of stylet 1 after the stylet second 12 is modified as described above.

As utilized herein, the terms “approximately,” “about,” “substantially” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described without restricting the scope of these features to the precise numerical ranges provided. Accordingly, these terms should be interpreted as indicating that insubstantial or inconsequential modifications or alterations of the subject matter described and are considered to be within the scope of the disclosure.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

For the purpose of this disclosure, the term “coupled” means the joining of two members directly or indirectly to one another. Such joining may be stationary or moveable in nature. Such joining may be achieved with the two members or the two members and any additional intermediate members being integrally formed as a single unitary body with one another or with the two members or the two members and any additional intermediate members being attached to one another. Such joining may be permanent in nature or may be removable or releasable in nature.

It should be noted that the orientation of various elements may differ according to other exemplary embodiments, and that such variations are intended to be encompassed by the present disclosure. It is recognized that features of the disclosed embodiments can be incorporated into other disclosed embodiments.

It is important to note that the constructions and arrangements of needle apparatuses or the components thereof as shown in the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter disclosed. For example, elements shown as integrally formed may be constructed of multiple parts or elements, the position of elements may be reversed or otherwise varied, and the nature or number of discrete elements or positions may be altered or varied. The order or sequence of any process or method steps may be varied or re-sequenced according to alternative embodiments. Other substitutions, modifications, changes and omissions may also be made in the design, operating conditions and arrangement of the various exemplary embodiments without departing from the scope of the present disclosure.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, describes techniques, or the like, this application controls.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, the technology described herein may be embodied as a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

1. A needle apparatus comprising:
a hollow tube component including, at a first end of the hollow tube component, a pointed portion configured for piercing tissue of a patient and a needle hub disposed at a second end of the hollow tube component, the hollow tube component including a bend disposed therein, the bend positioned between the first and second ends of the hollow tube component; and
a stylet removably positioned in the hollow tube component, the stylet including a proximal end, a distal end, and a stylet hub coupled to the distal end, wherein at least a portion of the proximal end of the stylet includes a first diameter that is smaller than a second diameter of at least a portion of the distal end of the stylet.

2. The apparatus of claim 1, wherein the at least the portion of the proximal end of the stylet including the first diameter has a length greater than a distance from the pointed portion of the hollow tube component to the bend.

3. The apparatus of claim 1, wherein the at least the portion of the proximal end of the stylet including the first diameter has a length of at least a distance from the pointed portion of the hollow tube component to the bend.

4. The apparatus of claim 1, wherein the second diameter corresponds to an inner diameter of the hollow tube component.

5. The apparatus of claim 1, wherein the stylet includes a pointed tip positioned at the proximal end.

6. The apparatus of claim 1, wherein the stylet is composed of stainless steel.

7. The apparatus of claim 1, wherein an inner diameter of the hollow tube component is substantially constant from the first end to the second end.

8. The apparatus of claim 1, wherein a diameter of the hollow tube component is smaller at the first end than at the second end.

9. The apparatus of claim 1, wherein the hollow tube component and the stylet are composed of the same material.

10. The apparatus of claim 1, wherein the hollow tube component and the stylet are composed of distinct materials.

11. A method of implementing a needle apparatus, the method comprising:
positioning a stylet in a tubular needle component, the stylet including a proximal end, a distal end and a stylet hub coupled to the distal end, wherein at least a portion of the proximal end of the stylet includes a first diameter that is smaller than a second diameter of at least a portion of the distal end of the stylet, the tubular needle component including, at a first end of the tubular needle component, a pointed portion configured for piercing tissue of a patient and, and a needle hub disposed at a second end of the tubular needle component, the tubular needle component including a bend disposed therein, the bend positioned between the first and second end of the tubular needle component; and
removing the stylet from the tubular needle component.

12. The method of claim 11, wherein the at least the portion of the proximal end of the stylet including the first diameter has a length greater than a distance from the pointed portion of the hollow tube component to the bend.

13. The method of claim 11, wherein the second diameter corresponds to an inner diameter of the hollow tube component.

14. The method of claim 11, wherein the stylet is composed of steel.

15. The method of claim 11, wherein an inner diameter of the hollow tube component is substantially constant from the first end to the second end.

16. The method of claim 11, wherein the hollow tube component and the stylet are composed of the same material.

17. A method of manufacturing a stylet for a needle apparatus, the method comprising:
removing material from the stylet such that at least a portion of a proximal end of the stylet includes a first diameter that is smaller than a second diameter of at least a portion of a distal end of the stylet; and
coupling a stylet hub to the distal portion of the stylet.

18. The method of claim 17, wherein the material is removed by at least one of grinding, milling, and electrical discharge machining

19. The method of claim 17, further comprising forming a pointed tip on the proximal end of the stylet.

20. The method of claim 17, wherein material is removed from the stylet such that the at least the portion of the proximal end of the stylet including the first diameter has a length greater than a distance from a pointed portion of a hollow tube of the needle apparatus to a bend in the hollow tube of the needle apparatus.
